(19) United States
(12) Patent Application Publication
(10) Pub. No.: US 2016/0235791 A1
(43) Pub. Date: Aug. 18, 2016

(54) BACTERIA OF THE STENOTROPHOMONAS GENUS AND/OR GROWTH INDUCER OF SAID BACTERIA FOR PREVENTING AND/OR TREATING ATOPIC DERMATITIS

(71) Applicant: L'OREAL, Paris (FR)

(72) Inventors: Richard Martin, Rochecorbon (FR); Sophie Seite, Paris (FR); Anne Colonna, Paris (FR)

(21) Appl. No.: 15/024,620

(22) PCT Filed: Sep. 25, 2014
(86) PCT No.: PCT/EP2014/070578

§ 371 Date: Mar. 24, 2016

(30) Foreign Application Priority Data
Sep. 25, 2013 (FR) 13 59246

(51) Int. Cl. A61K 35/74; C12Q 1/68; C12N 1/20; G01N 33/569; A61K 8/99; A61Q 19/00

(52) U.S. Cl. CPC A61K 35/74; A61K 8/99; A61Q 19/007; C12N 1/20; G01N 33/56911; C12Q 1/689; C12Q 2600/118; G01N 2333/195; G01N 2800/202; G01N 2800/50; G01N 2800/52

(57) ABSTRACT

This invention relates to a dermatological composition comprising, as an active ingredient, bacteria of at least one Stenotrophomonas strain and/or at least one Stenotrophomonas growth inducer, and the use thereof in the prevention and/or treatment of atopic dermatitis. This invention also relates to an in vitro method for prognosis and/or diagnosis of atopic dermatitis and a method for selecting a Stenotrophomonas growth inducer.

### FIELD OF THE INVENTION

This invention relates to bacteria of the Stenotrophomonas genus and/or a growth inducer of bacteria of the Stenotrophomonas genus for the prevention and/or treatment of atopic dermatitis.

### PRIOR ART

At present, more than 500 bacterial species have been detected on healthy skins, involving more than 2 million of genes. Around 10⁶ bacteria inhabit every cm² of skin. The microbiome of the skin of healthy subjects has been described as having a specificity according to 3 types of areas: moist, dry and sebaceous.

The bacterial ecosystem of the skin acts on the immune response and contributes to clinical signs of certain cutaneous imbalances, such as atopic dermatitis.

Atopic dermatitis, also called atopic eczema, is a chronic and inflammatory disease of the skin. Atopic dermatitis preferentially concerns infants and children, but also affects adolescents and adults. The number of patients with atopic dermatitis is constantly increasing.

Atopic dermatitis is characterized by cutaneous dryness, pruritus and/or eczema, these three symptoms generally appearing in succession. In fact, the first sign of atopic dermatitis is very dry skin, followed by the appearance of erythema and edemas, then blisters and scabby and oozing lesions. Moreover, atopic dermatitis is a disease that occurs in flare-ups.

The prevention of atopic dermatitis consists in hydrating the skin in people at risk or having already experienced one or more episodes of atopic dermatitis. The treatment of atopic dermatitis consists in hydrating the skin, reducing inflammation and relieving itching. In this regard, the application of hydrating creams and the oral or topical administration of antihistamines and/or anti-inflammatory drugs, such as cortisone, are prescribed. In flare-ups, it is important to monitor the appearance of bacterial secondary infections in lesion areas scratched by the patient. Atopic dermatitis is also a cause of sleeping problems due to the significant associated itching.

The existing treatments for atopic dermatitis are not always effective and flare-ups often occur when the treatment is stopped.

There is therefore still a need for alternative solutions for the prevention and/or treatment of atopic dermatitis.

### DETAILED DESCRIPTION

This invention is based on the demonstration of the involvement of bacteria of the Stenotrophomonas genus in atopic dermatitis.

The inventors in fact demonstrated a significant reduction in the number of bacteria of the Stenotrophomonas genus in lesion areas of patients with atopic dermatitis with respect to non-pathological areas in the same patients.

In addition, the inventors demonstrated that the treatment of lesion areas of patients with atopic dermatitis with a La Roche Posay thermal spring water-based product enables to significantly increase the number of bacteria of the Stenotrophomonas genus at lesion areas.

The inventors also demonstrated that the treatment of lesion areas with the La Roche Posay thermal spring water-based product enables to promote the proportion of bacteria of the Stenotrophomonas genus in the microbiome of the treated lesion area.

Interestingly, La Roche Posay thermal spring water naturally contains Stenotrophomonas maltophilia bacteria.

Using bacteria of the Stenotrophomonas genus and/or at least one growth inducer of bacteria of the Stenotrophomonas genus, this invention thus enables to restore the equilibrium of the microbiome with bacteria of the Stenotrophomonas genus in treated lesion areas.

This invention thus relates to a dermatological composition comprising, as an active ingredient, bacteria of at least one strain of the Stenotrophomonas genus and/or at least one growth inducer of bacteria of the Stenotrophomonas genus.

This invention also relates to bacteria of at least one strain of the Stenotrophomonas genus and/or a growth inducer of bacteria of the Stenotrophomonas genus, or a dermatological composition comprising them, for use in the prevention and/or treatment of atopic dermatitis.

Another object of the invention concerns an in vitro method for prognosis and/or diagnosis of atopic dermatitis comprising the steps consisting in:
a) measuring the level of bacteria of the Stenotrophomonas genus in a sample from an area suspected to be a lesion area or an area suspected to become a lesion area in an individual, and
b) deducing, from the measurement obtained in step a), whether the individual is at risk of having or has atopic dermatitis.

Yet another object of the invention concerns a method for selecting a growth inducer of bacteria of the Stenotrophomonas genus, comprising the steps consisting in:
placing a compound to be tested in contact with bacteria of the Stenotrophomonas genus,
measuring the growth of bacteria in the presence of said compound to be tested, and
selecting, as a growth inducer of bacteria of the Stenotrophomonas genus, a compound capable of improving the growth of bacteria of the Stenotrophomonas genus with respect to their growth in the absence of said compound.

Definitions—Prevention and/or Treatment of Atopic Dermatitis

This invention relates primarily to the prevention and/or treatment of atopic dermatitis in humans.

By the expression “in humans”, it is herein meant males and females of any age, in particular infants, children, adolescents, adults and elderly people.

A human being will hereafter be referred to by the terms patient(s) or individual(s).

Atopic dermatitis is described as being associated with a deficit in stratum corneum lipid metabolism, and in particular ceramide metabolism. This pathology appears in the form of more or less chronic xerosis affecting a large span of the body, associated with inflammatory and pruritic flare-ups in plaques.

The term “xerosis” means drying of the skin, also called cutaneous dryness.

“Atopy” is a hereditary predisposition of the immune system to prefer hypersensitivity reactions mediated by immunoglobulins E (IgE) with respect to common antigens in the diet, the outside or domestic environment.

Patients with atopic dermatitis have a so-called atopic skin.

The term “skin” herein encompasses both the skin and the scalp, but does not comprise mucous membranes.

The terms “area”, “skin area” and “cutaneous area” are herein used interchangeably.

By the term “lesion area”, it is herein meant an area of the skin affected by atopic dermatitis.

The lesion areas are in particular characterized by skin dryness, redness, blisters, scabs or combinations thereof.

The inventors have demonstrated that lesion areas are also characterized by a microbiome of low diversity, comprising predominantly bacteria of the Staphylococcus genus and a lower proportion of bacteria of the Stenotrophomonas genus than in the microbiome of non-lesion areas.

Inflammation, pruritus and intense dryness are three characteristic symptoms of atopic dermatitis.

The terms “pruritus” and “itching” are herein used interchangeably.

By the term “microbiome”, it is herein meant all genomes of bacteria present at the surface of a cutaneous area of a human being.

By contrast with a “lesion area”, the terms “non-lesion area”, “non-pathological area” or “healthy area” refer to an area of the skin not affected by atopic dermatitis, and preferably not affected by any other pathology or cutaneous wound.

By “area suspected to be a lesion area” , it is meant, for example, an area of the skin having at least one characteristic selected from the group consisting of an inflammation, a pruritus, a scabby lesion and an oozing lesion, preferably at least two of these characteristics.

By “area suspected to become a lesion area”, it is meant, for example, an area having xerosis and/or a skin area having been a lesion area.

By the expression “method for diagnosis of atopic dermatitis”, it is herein meant a method enabling to determine whether an individual has atopic dermatitis.

By the expression “method for prognosis of atopic dermatitis”, it is herein meant a method enabling to determine whether an individual is at risk of having atopic dermatitis.

By the expression “prevention of atopic dermatitis”, it is meant the prophylactic or preventive treatment of atopic dermatitis, which consists in preventing the appearance of atopic dermatitis, and in particular xerosis.

By the expression “treatment of atopic dermatitis”, it is meant the therapeutic treatment of atopic dermatitis, which consists in reducing, inhibiting, eliminating symptoms of atopic dermatitis, such as xerosis, inflammation, eczema and pruritus, the span of lesions, or the progression of atopic dermatitis, for example by delaying, spacing apart or suppressing inflammatory and pruritic flare-ups.

Bacteria of the Stenotrophomonas Genus

The bacteria of the Stenotrophomonas genus, also called stenotrophomonas, are aerobic gram-negative proteobacteria.

The strain of the Stenotrophomonas genus is, for example, selected from the group consisting of Stenotrophomonas acidaminiphila, Stenotrophomonas africana, Stenotrophomonas chelatiphaga, Stenotrophomonas daejeonensis, Stenotrophomonas dokdonensis, Stenotrophomonas ginsengisoli, Stenotrophomonas humi, Stenotrophomonas koreensis, Stenotrophomonas maltophilia, Stenotrophomonas nitritireducens, Stenotrophomonas pavanii, Stenotrophomonas rhizophila, and Stenotrophomonas terrae.

The strain of the Stenotrophomonas genus according to the invention is preferably a strain naturally existing at the surface of the skin of a human being.

A preferred strain of the Stenotrophomonas genus according to the invention is a Stenotrophomonas maltophilia strain.

The bacteria of the Stenotrophomonas genus may be in living form, inactivated form, attenuated form, in the form of a fraction of said bacteria, for example a lipopolysaccharide (LPS) of said bacteria, or in a combination of said forms.

The bacteria of the Stenotrophomonas genus used in the context of this invention are preferably in inactivated form.

The terms “in inactivated form”, “in non-revivable form” and “in dead form” are herein synonymous.

Bacteria “in attenuated form” are bacteria that have partially or entirely lost their possibly pathogenic properties.

The bacteria used in the context of this invention may be bacteria from one or more strains of the Stenotrophomonas genus.

When the bacteria come from one or more strains of the Stenotrophomonas genus, they are strains of the same species and/or strains of different species.

By “bacteria of a strain of the Stenotrophomonas genus” or “bacteria from a strain of the Stenotrophomonas genus”, it is herein meant bacteria obtained by culture of a strain of the Stenotrophomonas genus.

A preferred strain of the Stenotrophomonas genus according to the invention is provided in the form of La Roche Posay thermal spring water and/or an extract of La Roche Posay thermal spring water.

La Roche Posay thermal spring water is extracted from the spring with the same name. It is a bicarbonated, calcic, silicated and selenitic water. La Roche Posay thermal spring water has a total carbonate and bicarbonate concentration above 360 mg/l and a mineralization greater than or equal to 400 mg/l. Preferably, La Roche Posay thermal spring water comprises at least 9 mg/l of silicon oxide.

Typically, La Roche Posay thermal spring water comprises between 370 mg/l and 410 mg/l, preferably between 380 mg/l and 400 mg/l of bicarbonate ions, between 120 mg/l and 160 mg/l, preferably between 130 mg/l and 150 mg/l of calcium ions, and at least 4 mg/l of sulfates.

For example, La Roche Posay thermal spring water comprises 387 mg/l of bicarbonate ions, 140 mg/l of calcium ions and at least 4 mg/l of sulfates.

Growth Inducer of Bacteria of the Stenotrophomonas Genus

A growth inducer of bacteria of the Stenotrophomonas genus is a compound that enables to improve the growth of bacteria of the Stenotrophomonas genus, in particular Stenotrophomonas maltophilia, with respect to the growth obtained in the absence of said growth inducer.

By “improve the growth of bacteria”, it is in particular meant the fact that the growth inducer of bacteria of the Stenotrophomonas genus enables bacteria of the Stenotrophomonas genus to increase their proportion in a given microbiome.

The growth inducer of bacteria of the Stenotrophomonas genus may, for example, enable faster multiplication of bacteria of the Stenotrophomonas genus with respect to the multiplication of other bacteria present in the microbiome.

In a preferred embodiment, the growth inducer of bacteria of the Stenotrophomonas genus is selected from the group consisting of one or more keratins, one or more keratin derivatives, one or more yeast peptones, one or more yeast extracts, one or more sugars, one or more trace elements and combinations thereof.

By “keratin derivative”, it is herein meant partially or completely hydrolyzed keratins.

The growth inducer is preferably used in a composition in which the saline, in particular NaCl, concentration is below 6 g/l, preferably below 3 g/l.

A preferred growth inducer of bacteria of the Stenotrophomonas genus according to the invention is provided in the form of La Roche Posay thermal spring water and/or an extract of La Roche Posay thermal spring water.

Composition

This invention relates in particular to a dermatological composition comprising, as an active ingredient, bacteria of at least one strain of the Stenotrophomonas genus and/or at least one growth inducer of bacteria of the Stenotrophomonas genus.

Said composition is a dermatological composition or a cosmetic composition.

A preferred composition according to the invention is a dermatological composition.

The strain of the Stenotrophomonas genus, in particular Stenotrophomonas maltophilia, and the growth inducer of bacteria of the Stenotrophomonas genus, in particular Stenotrophomonas maltophilia, are in particular as defined above.

The composition according to the invention may also comprise other microorganisms, for example chosen among the microorganisms useful in the prevention and/or treatment of atopic dermatitis.

These other microorganisms may be present in the composition in living form, in vegetative or sporulated state, or in inactive form.

In another embodiment, the composition according to the invention does not comprise any microorganism other than bacteria of the Stenotrophomonas genus or does not comprise any microorganism other than Stenotrophomonas maltophilia.

The composition according to the invention may comprise one or more growth inducers of bacteria of the Stenotrophomonas genus, for example at least two or at least three growth inducers of bacteria of the Stenotrophomonas genus.

The composition according to the invention may advantageously comprise both bacteria of at least one strain of the Stenotrophomonas genus and at least one growth inducer of bacteria of the Stenotrophomonas genus, said growth inducer preferably improving the growth of bacteria of the strain(s) of the Stenotrophomonas genus present in said composition.

When the composition according to the invention comprises bacteria of a strain of the Stenotrophomonas genus, said composition may comprise 100 to 1000000 bacterial cell equivalents of bacteria of said strain of the Stenotrophomonas genus for 100 g of said composition, preferably 10000 to 100000 bacterial cell equivalents for 100 g of said composition.

When the composition according to the invention comprises a growth inducer of bacteria of the Stenotrophomonas genus, said composition may comprise 0.01% to 5% of said growth inducer, preferably 0.1% to 1% of said growth inducer, the percentages being expressed in g for 100 g of said composition.

The composition according to the invention may also advantageously comprise at least one sugar, for example a hexose such as glucose.

In a preferred embodiment, the composition according to the invention has a saline, in particular NaCl, concentration of below 6 g/l, preferably below 4 g/l, and more preferentially below 3 g/l.

In a preferred embodiment according to the invention, the composition is characterized in that the bacteria of at least one strain of the Stenotrophomonas genus, preferably at least one strain of Stenotrophomonas maltophilia, and/or said growth inducer of bacteria of the Stenotrophomonas genus are provided in the form of La Roche Posay thermal spring water and/or an extract of La Roche Posay thermal spring water.

In a preferred embodiment, the composition according to the invention does not comprise any microorganism other than those provided by the La Roche Posay thermal spring water and/or an extract of La Roche Posay thermal spring water.

This invention relates in particular to a composition as defined above, characterized in that it comprises 1% to 80% of La Roche Posay thermal spring water, preferably 20% to 60%, more preferentially 30% to 50%, the percentages being expressed in g for 100 g of composition.

In another embodiment, the composition does not contain La Roche Posay thermal spring water and/or an extract of La Roche Posay thermal spring water.

In another embodiment, this invention thus relates to a composition comprising, as an active ingredient, bacteria of at least one strain of the Stenotrophomonas genus and/or at least one growth inducer of bacteria of the Stenotrophomonas genus, said composition not comprising La Roche Posay thermal spring water and/or not comprising an extract of La Roche Posay thermal spring water. In this case, the bacteria of at least one strain of the Stenotrophomonas genus and/or the growth inducer(s) of bacteria of the Stenotrophomonas genus are provided in another form.

The composition according to the invention is preferably suitable for topical application.

The terms “topical” and “on the skin” are herein synonymous.

The composition can thus be in any form suitable for topical use, such as a cream, a balm, a gel, an oil, a water, a pomade, a paste or a spray.

Method for Preventing and/or Treating Atopic Dermatitis

This invention also relates to bacteria of at least one strain of the Stenotrophomonas genus and/or at least one growth inducer of bacteria of the Stenotrophomonas genus, for use in the prevention and/or treatment of atopic dermatitis.

In a preferred embodiment, the bacteria of at least one strain of the Stenotrophomonas genus and/or the growth inducer(s) of bacteria of the Stenotrophomonas genus are formulated in a dermatological composition.

The strain of the Stenotrophomonas genus, in particular Stenotrophomonas maltophilia, the growth inducer of bacteria of the Stenotrophomonas genus, in particular Stenotrophomonas maltophilia, and the dermatological composition are in particular as defined above.

The dermatological composition is typically applied one to three times per day, for example two times per day, on lesion areas and preferably also around lesion areas, and/or on areas suspected to become lesion areas.

The treatment concerns patients with atopic dermatitis.

The prevention concerns individuals at risk of having atopic dermatitis, such as individuals having had at least one episode of atopic dermatitis in their life or having significant cutaneous dryness.

This invention relates in particular to bacteria of at least one strain of the Stenotrophomonas genus, a growth inducer of bacteria of the Stenotrophomonas genus, or a dermatological composition comprising bacteria of at least one strain of the Stenotrophomonas genus and/or at least one growth inducer of bacteria of the Stenotrophomonas genus, for use in the prevention and/or treatment of atopic dermatitis in individuals diagnosed with atopic dermatitis or at risk of having atopic dermatitis, in particular after implementation of the method for prognosis and/or diagnosis according to the invention, described below.

The treatment is performed at least until the symptoms disappear.

The treatment is preferably continued for several days or several weeks after the disappearance of symptoms, possibly with a gradual reduction in the frequency of administration of the dermatological composition.

This invention also relates to a method for preventing and/or treating atopic dermatitis comprising the administration of a dermatological composition according to the invention on the skin, at lesion areas and preferably also around lesion areas, and/or on areas suspected to become lesion areas.

This invention also relates to bacteria of at least one strain of the Stenotrophomonas genus and/or at least one growth inducer of bacteria of the Stenotrophomonas genus for their use in the prevention and/or treatment of atopic dermatitis as defined above, characterized in that said bacteria and/or said growth inducer enables to balance the proportion of bacteria of the Stenotrophomonas genus within the microbiome present at the skin surface, preferably at a lesion area on the skin or an area suspected to become a lesion area, in an individual with atopic dermatitis or at risk of having atopic dermatitis.

This invention also relates to a method intended to balance the proportion of bacteria of the Stenotrophomonas genus in the microbiome present at the skin surface, comprising the topical administration of bacteria of at least one strain of the Stenotrophomonas genus and/or at least one growth inducer of bacteria of the Stenotrophomonas genus and/or a dermatological composition as defined above, in an individual with atopic dermatitis or at risk of having atopic dermatitis, preferably at a lesion area of the skin and/or an area suspected to become a lesion area.

Method of Cosmetic Treatment

This invention also relates to a cosmetic treatment method comprising a step of applying on dry skin, in particular in a subject not having atopy, a cosmetic composition comprising at least one strain of the Stenotrophomonas genus and/or at least one growth inducer of bacteria of the Stenotrophomonas genus.

The cosmetic composition is in particular as defined above.

This invention also relates to the use of a cosmetic composition comprising at least one strain of the Stenotrophomonas genus and/or at least one growth inducer of bacteria of the Stenotrophomonas genus in order to soothe dry skin, in particular in an individual without atopy.

An individual without atopy has a so-called non-atopic skin.

In vitro Method for Prognosis and/or Diagnosis

When the population of bacteria of the Stenotrophomonas genus decreases or begins to decrease before the appearance of symptoms of atopic dermatitis, it is thus possible to prevent inflammatory and pruritic flare-ups even though clinical symptoms have not appeared.

This invention thus also relates to an in vitro method for prognosis and/or diagnosis of atopic dermatitis comprising steps consisting in:
a) measuring the level of bacteria of the Stenotrophomonas genus in a sample coming from an area suspected to be a lesion area or suspected to become a lesion area of an individual,
b) deducing, from the measurement obtained in step a) whether the individual has a risk of having or has atopic dermatitis.

By “level of bacteria”, it is for example meant the amount or concentration of bacteria.

The level of bacteria may be measured by any suitable method well known to a person skilled in the art, such as measurement of bacterial DNA, bacterial RNA (in particular RNA16S), the signal obtained after labeling of bacteria, for example with an antibody, or by ELISA (Enzyme Linked ImmunoSorbant Assay) (in particular by using an antibody onto which an enzymatic system enabling development of a color in the visible is grafted).

The in vitro method for prognosis and/or diagnosis of atopic dermatitis may also comprise, between step a) and step b), a step consisting in comparing the level of bacteria measured in step a) with the level of bacteria of the Stenotrophomonas genus in a sample from a non-lesion area of the same patient and/or from a non-pathological area of an individual not suffering from atopic dermatitis, and/or with a reference value.

The reference value may be obtained from a mean of levels of bacteria of the Stenotrophomonas genus measured in samples from several individuals from a non-lesion area of patients with atopic dermatitis and/or from a non-pathological area of an individual not suffering from atopic dermatitis.

A sample from a given skin area is, for example, obtained by applying on said skin area an adhesive disc, for example a disc of the type D-Squame, or by scratching the surface of said skin area.

The area of the sample has a surface of 0.2 cm² to 3 cm², preferably 0.5 cm² to 2 cm².

In a preferred embodiment, the non-lesion area is a healthy area that is as close as possible to the lesion area.

A non-lesion area is thus preferably located near the lesion area, for example at a distance of at least 0.2 cm, at least 0.4 cm, at least 0.6 cm, at least 0.8 cm or at least 1 cm from the edges of the lesion area, and preferably at a distance of at most 3 cm or at most 2 cm from the edges of the lesion area.

Method for Selecting a Growth Inducer of Bacteria of the Stenotrophomonas Genus

This invention also relates to a method for selecting a growth inducer of bacteria of the Stenotrophomonas genus, comprising steps consisting in:
placing a compound to be tested in contact with bacteria of the Stenotrophomonas genus,
measuring the growth of bacteria in the presence of said compound to be tested, and
selecting, as a growth inducer of bacteria of the Stenotrophomonas genus, a compound capable of improving the growth of bacteria with respect to their growth in the absence of said compound.

The compound to be tested may, for example, be a La Roche Posay water extract.

Said selection method is an in vitro or ex vivo method.

In an advantageous embodiment, the step of placing the compound to be tested in contact with bacteria of the Stenotrophomonas genus is performed in a microbiome.

This microbiome may be a microbiome of an individual or may be a medium comprising the different bacteria in proportions corresponding to a reference microbiome.

The microbiome of said individual and/or the reference microbiome may be a microbiome of a lesion area of a patient with atopic dermatitis or of a non-lesion area of a patient with atopic dermatitis or of an individual not suffering from atopic dermatitis.

The reference microbiome may also be a medium comprising different bacteria in different proportions, which does not necessarily correspond to a natural microbiome.

The microbiome of said individual and/or the reference microbiome is preferably a microbiome of a lesion area of a patient with atopic dermatitis, more preferentially an untreated lesion area.

This invention thus relates to a method for selecting a growth inducer of bacteria of the Stenotrophomonas genus, comprising the steps consisting in:
placing a compound to be tested in contact with bacteria of the Stenotrophomonas genus within a microbiome,
measuring the growth of bacteria of the Stenotrophomonas genus in the presence of said compound to be tested, and
selecting, as a growth inducer of bacteria of the Stenotrophomonas genus, a compound capable of improving the growth of bacteria of the Stenotrophomonas genus with respect to their growth in the absence of said compound, in particular a compound capable of increasing the proportion of bacteria of the Stenotrophomonas genus within the microbiome.

In another advantageous embodiment, the step of placing the compound to be tested in contact with bacteria of the Stenotrophomonas genus is performed in a medium comprising bacteria of the Stenotrophomonas genus as the only microorganism. In this case, the selection method preferably comprises an ulterior step of placing the compound to be tested improving the growth of bacteria of the Stenotrophomonas genus present as the only microorganism, with the bacteria of the Stenotrophomonas genus within a microbiome, the microbiome being as defined above.

This invention thus relates to a method for selecting a growth inducer of bacteria of the Stenotrophomonas genus, comprising the steps consisting in:
placing a compound to be tested in contact in a medium comprising bacteria of the Stenotrophomonas genus as the only microorganism,
measuring the growth of bacteria of the Stenotrophomonas genus in the presence of said compound to be tested,
selecting a compound capable of improving the growth of bacteria of the Stenotrophomonas genus with respect to their growth in the absence of said compound in said medium,
preferably, placing the compound selected in the previous step in contact with bacteria of the Stenotrophomonas genus within a microbiome,
preferably, measuring the growth of bacteria of the Stenotrophomonas genus in the presence of said compound within said microbiome, and
selecting, as a growth inducer of bacteria of the Stenotrophomonas genus, a compound capable of improving the growth of bacteria of the Stenotrophomonas genus with respect to their growth in the absence of said compound, in particular a compound capable of increasing the proportion of bacteria of the Stenotrophomonas genus within the microbiome.

The term “medium” refers to a medium suitable for the growth of bacteria.

It may be a solid or a liquid medium.

The selection method is carried out under conditions enabling the growth of bacteria in the absence of said compound to be tested.

Other features and advantages of the invention will become clear from the following examples, provided as illustrative and non-limiting examples.

### FIGURES

FIG. 1: Mean relative abundance by genus of bacterium at lesion and non-lesion areas in the same individual with atopic dermatitis, before treatment and after treatment. For each genus, the 1^{st} column corresponds to the results on the non-lesion area before treatment, the 2^{nd} column corresponds to the non-lesion area after treatment, the 3^{rd} column corresponds to the lesion area before treatment and the 4^{th} column corresponds to the lesion area after treatment.

FIG. 2: Mapping of the Staphylococcus genus by species at the lesion and non-lesion areas in the same individual with atopic dermatitis, before treatment and after treatment. On the y-axis is the mean relative abundance (1 represents 100%). A: Staphylococcus epidermidis, B: Staphylococcus aureus, C: Staphylococcus spp., D: Staphylococcus haemolyticus, E: other Staphylococcaceae, 1: non-lesion area before treatment, 2: non-lesion area after treatment, 3: lesion area before treatment, 4: lesion area after treatment.

FIG. 3: Photograph of the bacterial diversity by genus on non-atopic skin (mean obtained on arms of 93 non-atopic females).

FIG. 4: Photograph of the bacterial diversity by genus at 84 days after the start of treatment on lesion areas of patients with atopic dermatitis and responding favorably to treatment.

FIG. 5: Photograph of the bacterial diversity by genus at 84 days after the start of treatment on non-lesion areas of patients with atopic dermatitis and responding favorably to treatment.

### EXAMPLE

### Material and Methods

Patients

The study was conducted on 50 patients with atopic dermatitis and having lesion areas and non-lesion areas. The mean SCORAD (overall severity score of atopic dermatitis) at inclusion was 33±5.6. The mean age was 12±9 years.

Procedure

Les 50 patients applied the La Roche Posay thermal spring water-based product of La Roche Posay “Baume Lipikar AP” for 84 days, at a frequency of two applications per day.

Several bacterial samples are taken on lesion areas and neighboring non-lesion areas of each subject, in the period ranging from before the start of treatment until the end of treatment.

Photographs of the sampled areas are taken.

The dryness, erythema and desquamation of the areas sampled are evaluated.

The size of the sample area is small, in particular from 0.5 to 2 cm². The area sampled is most representative of the lesion area.

The sampling is performed with sterile single-use cotton swabs under a sterile air flow. The sterile air flow is produced by a portable laminar flow hood. It filters the ambient air and enables a so-called “axenic” sample, i.e. without microorganisms from the environment. The sampler never cuts off the laminar air flow during the sampling. The sampling is performed in a quiet location and both the sampled subject and the sampler are silent during the operation. The sterile cotton swab is immersed in a milliQ water solution containing 0.15 M NaCl and 0.1% Tween 20. This solution is filtered to 0.22 μm (Minisart ref. 16534) extemporaneously under the laminar flow hood so as to ensure its sterility. The sample was standardized on the basis of the area sampled (force, time and surface). The cotton swab is broken in an Eppendorf tube sterilized at 121° C./30′ guaranteed to be free of RNAse and DNAse. The samples are immediately placed at −20° C. for several hours, then transferred to −80° C. (preferably directly placed at −80° C.) where they are stable for at least 6 months. After amplification of the bacterial DNA coding for the RNA 16S, both the bacterium genus and species are determined, by analysis using the Qiime bacterial database.

The comparison of the microbiome is performed on a lesion and non-lesion area of the same subject. The diversity of the microbiome is analyzed by a plurality of methods, comprising the Shannon Index, which is the one most commonly used by the person skilled in the art. The Shannon Index is obtained by the following formula:

$${H}^{′} = - {\sum }_{i = 1}^{s} {p}_{i} {log}_{2} {p}_{i}$$

H′: Shannon biodiversity index
i: a species from the study medium
pᵢ: Proportion of a species with respect to the total number of species (S) in the study medium (or specific richness of the medium), which is calculated as follows:

p(i)=nᵢ/N

where nᵢ is the number of individuals for the species i and N is the total number (the individuals of all of the species).

### Results

Clinical Analysis (n=50 Subjects)

At inclusion of the subjects, the overall severity score of atopic dermatitis (SCORAD) is not correlated with age or the sex of the individuals.

The age of the disease is strongly correlated with the age of the patient, which confirms the “innate” nature of the pathology (including a genetic component demonstrated with filaggrin polymorphisms) with some subjects, however, recently and severely affected (eczema acquired or cumulative with triggering environmental factors).

The severity of the erythema and the dryness of the lesions sampled at inclusion are factors well correlated (erythema p=3.10-9, dryness p=1.10-4) with the overall score of the patient.

In this study, the treatment reduces the overall atopy score more considerably in males than in females.

36 patients responded favorably to the treatment and 14 patients did not respond to the treatment, comprising one patient whose SCORAD worsened during the period.

Cutaneous Microflora Analysis

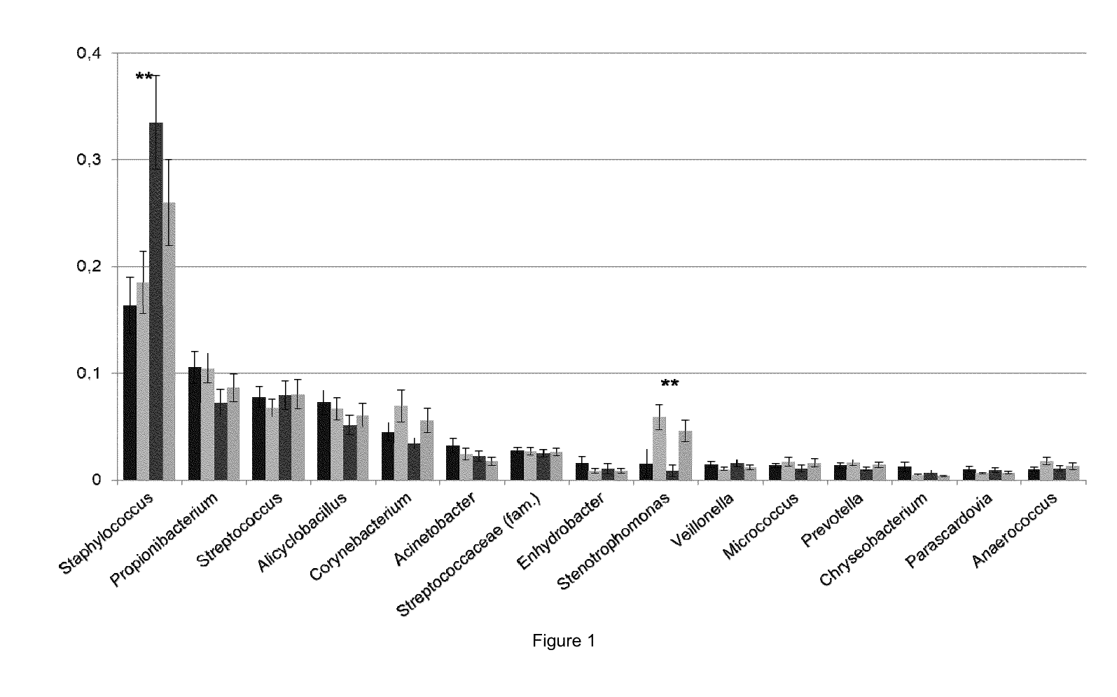

Before treatment, the microbiome of the lesion areas is significantly different from the adjacent non-lesion areas (p=0.001) (cf. FIG. 1).

The microbiome of the lesion areas has less bacterial diversity than that of the adjacent areas, richness measured by two reference indices (Shannon, p=0.002 and Bray-Curtis).

The diversity tends to disappear in favor of a common signature on lesion areas, independently of the area sampled.

After a treatment, the microbiome of the lesion areas evolves into a bacterial community similar to that of the adjacent non-lesion areas (p=0.16). This microbial community remains different from that observed before treatment, regardless of the areas (p=0.002). In other words, the microbiome of the non-lesion areas at inclusion is also modified as a result of the treatment.

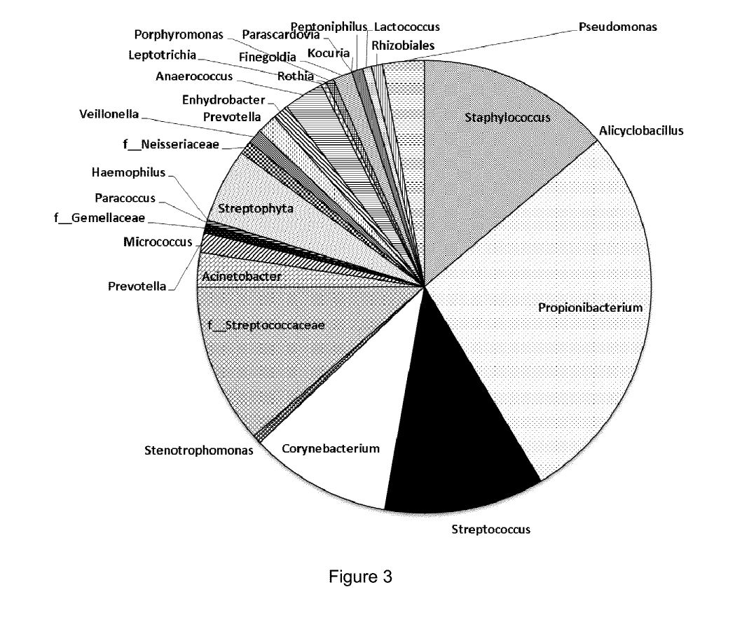

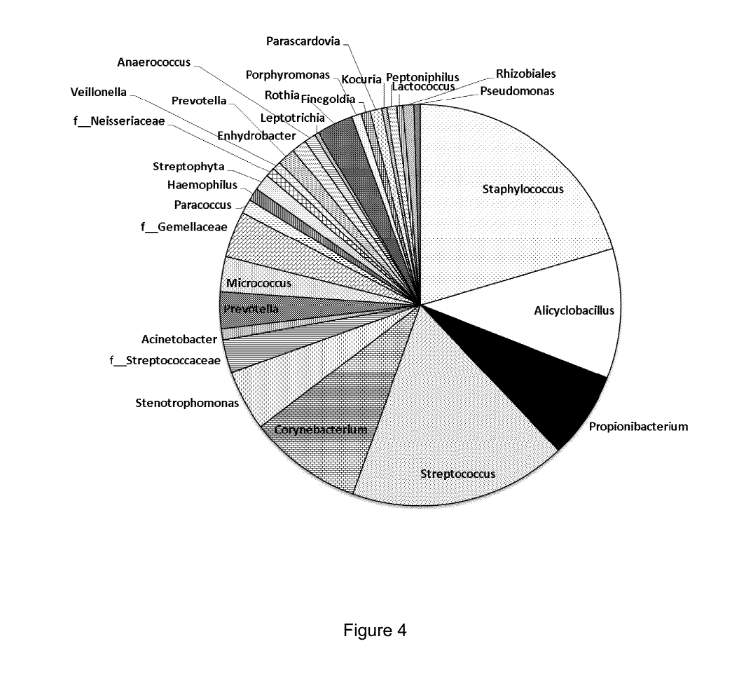

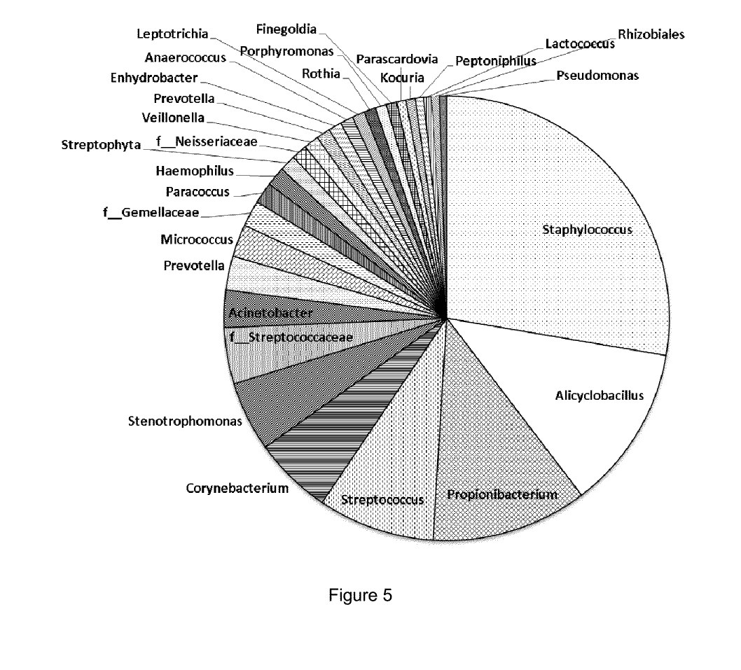

The treatment of the lesion area shows a significant enrichment in the Stenotrophomonas genus with respect to the untreated lesion areas (cf. FIGS. 1 and 3 to 5).

The mean value for 105 American females of Indo-European origin, in whom samples were taken from 7 areas of the body (scalp, forehead, cheeks, nose, armpit, arm and palm of the hand) shows that the percentage of Stenotrophomonas on the skin is close to 0.16% in non-atopic subjects.

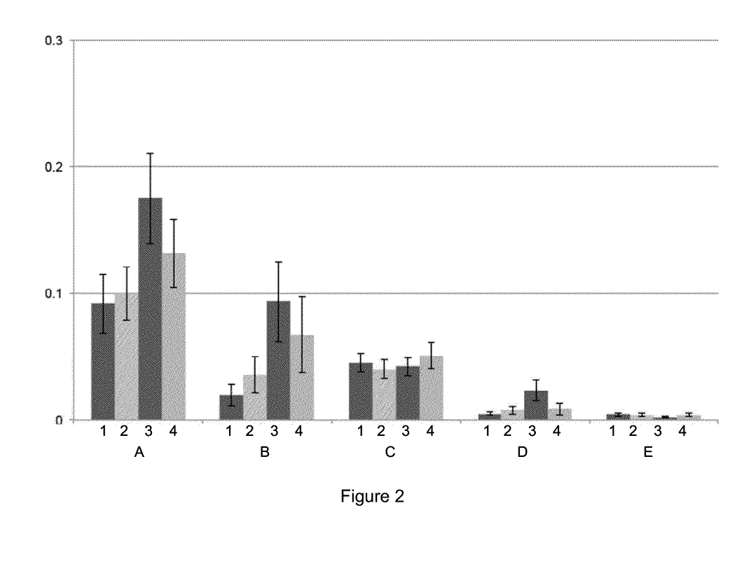

Staphylococci are the most represented genus of microflora in the lesion area and also in the adjacent non-lesion area (from 15 to 35% of the total microflora measured) (cf. FIGS. 1 and 2).

1. Dermatological composition comprising, as an active ingredient, bacteria of at least one strain of the Stenotrophomonas genus and/or at least one growth inducer of bacteria of the Stenotrophomonas genus.

2. Composition according to claim 1, which comprises 100 to 1000000 bacterial cell equivalents of bacteria of said strain of the Stenotrophomonas genus for 100 g of said composition.

3. Composition according to claim 1, wherein said Stenotrophomonas strain is selected from the group consisting of Stenotrophomonas acidaminiphila, Stenotrophomonas africana, Stenotrophomonas chelatiphaga, Stenotrophomonas daejeonensis, Stenotrophomonas dokdonensis, Stenotrophomonas ginsengisoli, Stenotrophomonas humi, Stenotrophomonas koreensis, Stenotrophomonas maltophilia, Stenotrophomonas nitritireducens, Stenotrophomonas pavanii, Stenotrophomonas rhizophila, and Stenotrophomonas terrae.

4. Composition according to claim 1, wherein said growth inducer is selected from the group consisting of one or more keratins, one or more keratin derivatives, one or more yeast peptones, one or more yeast extracts, one or more sugars, one or more trace elements, and combinations thereof.

5. Composition according to claim 1, which comprises 0.01% to 5% of said growth inducer, the percentages being expressed in g for 100 g of composition.

6. A method for the prevention and/or treatment of atopic dermatitis which comprises applying to a subject in need thereof bacteria of at least one strain of Stenotrophomonas and/or growth inducer of Stenotrophomonas.

7. The method according to claim 6, wherein said bacteria and/or said growth inducer are formulated in a dermatological composition.

8. The method according to claim 7, wherein said bacteria and/or said growth inducer are provided in the form of La Roche Posay thermal spring water or an extract of La Roche Posay thermal spring water.

9. The method according to claim 8, wherein the dermatological composition comprises 1% to 80% of La Roche Posay thermal spring water, the percentages being expressed in g for 100 g of composition.

10. The method according to claim 7, wherein the dermatological composition is suitable for topical use.

11. Method of cosmetic treatment comprising a step of applying on dry skin a cosmetic composition comprising at least one strain of the Stenotrophomonas genus and/or at least one growth inducer of bacteria of the Stenotrophomonas genus.

12. In vitro method for prognosis and/or diagnosis of atopic dermatitis comprising the steps consisting in:
a) measuring the level of bacteria of the Stenotrophomonas genus in a sample from an area suspected to be a lesion area or an area suspected to become a lesion area of an individual,
b) deducing from the measurement obtained in step a) whether the individual has a risk of having or has atopic dermatitis.

13. Method for selecting a growth inducer of bacteria of the Stenotrophomonas genus, comprising the steps consisting in:
placing a compound to be tested in contact with bacteria of the Stenotrophomonas genus,
measuring the growth of bacteria in the presence of said compound to be tested, and
selecting, as a Stenotrophomonas growth inducer, a compound capable of improving the growth of bacteria with respect to their growth in the absence of said compound.

14. Composition according to claim 2, wherein said Stenotrophomonas strain is selected from the group consisting of Stenotrophomonas acidaminiphila, Stenotrophomonas africana, Stenotrophomonas chelatiphaga, Stenotrophomonas daejeonensis, Stenotrophomonas dokdonensis, Stenotrophomonas ginsengisoli, Stenotrophomonas humi, tenotrophomonas koreensis, Stenotrophomonas maltophilia, Stenotrophomonas nitritireducens, Stenotrophomonas pavanii, Stenotrophomonas rhizophila, and Stenotrophomonas terrae.

15. Composition according to claim 2, wherein said growth inducer is selected from the group consisting of one or more keratins, one or more keratin derivatives, one or more yeast peptones, one or more yeast extracts, one or more sugars, one or more trace elements, and combinations thereof.

16. Composition according to claim 3, wherein said growth inducer is selected from the group consisting of one or more keratins, one or more keratin derivatives, one or more yeast peptones, one or more yeast extracts, one or more sugars, one or more trace elements, and combinations thereof.

17. Composition according to claim 2, which comprises 0.01% to 5% of said growth inducer, the percentages being expressed in g for 100 g of composition.

18. Composition according to claim 3, which comprises 0.01% to 5% of said growth inducer, the percentages being expressed in g for 100 g of composition.

19. Composition according to claim 4, which comprises 0.01% to 5% of said growth inducer, the percentages being expressed in g for 100 g of composition.

20. Composition according to claim 1, which comprises 0.1% to 1% of said growth inducer, the percentages being expressed in g for 100 g of composition.
